(19) United States
(12) Patent Application Publication
(10) Pub. No.: US 2012/0116205 A1
(43) Pub. Date: May 10, 2012

(54) IMAGING GUIDEWIRE

(75) Inventor: Arieh Sher, Petach Tikva (IL)

(73) Assignee: ARIOMEDICA LTD., Tel Aviv (IL)

(21) Appl. No.: 13/382,160

(22) PCT Filed: Jul. 26, 2010
(86) PCT No.: PCT/IB10/53372

§ 371 Date: Jan. 4, 2012

Related U.S. Application Data
(60) Provisional application No. 61/228,580, filed on Jul. 26, 2009.

(51) Int. Cl. A61B 6/00

(52) U.S. Cl. 600/407

(57) ABSTRACT

An imaging guidewire having at its distal tip at least a first Imaging sensor of a forward looking imaging system directed towards an area to be treated and configured to provide imaging data to a processing system, an optical imaging system directed towards an area that has already been treated and configured to provide imaging data of a treated area to the image processing system and at least one display device for displaying images processed by the image processing system. Operating the imaging guidewire during a medical procedure includes the steps of: Generating an image of an area to be treated; Upon completion of at least a portion of the medical treatment, generating an image of an area that has been treated; and displaying at least the first and second images; wherein each one of the first and second images can be generated by one or more imaging modalities.

### FIELD AND BACKGROUND OF THE INVENTION

This patent application relates to imaging guidewires or probes that include two or more imaging modalities so as to provide image data relating to two or more factors of a medical procedure.

My U.S. Pat. No.7,734,332 describes an atherectomy device with an imaging guidewire. The device is entitled ARIO (Apparatus for Removal of Intraluminal Occlusios). The device enables the physician to open any type of occlusion, both partially or totally occluded blood vessel and remove any type of plaque material or blood clots in a safe and non traumatic manner. To accomplish this goal the device includes three main elements: 1) Imaging guidewire; 2) Positioning balloons; 3) Powerful cutter.

It is stated in U.S. Pat. No. 7,734,332 that the imaging modality must generate a cross sectional view of the vessel. The importance of providing the cross sectional view of the vessel is that it enables the physician to make a decision of how to position the cutter in the lumen before it is operated, to reduce the risk of damage that could be caused to the vessel walls. If the cutter is positioned in the vessel in such a way that might damage the vessel wall the physician can re-position the cutter by controlling the positioning balloons. IVUS (Intravascular Ultrasound) or OCT (Optical Coherence Tomography) are currently used as imaging modalities that can provide a cross sectional view of the vessel. Both modalities have different characteristics.

IVUS has the ability to penetrate deeply through biological media such as blood and soft tissues (about 2 cm), but its resolution is about 100 um. Contrary to that; OCT generally has superior resolution to ultrasound (5-20 um) and has the potential to better identify some structures or components in vascular and other tissues, but its penetration depth is shallow and is about 1 mm

The advantages in combining these two modalities into one probe are known in the prior art. Tearney et al. describe in U.S. Pat. No. 6,134,003 an OCT imaging system into which an ultrasonic system is coupled (FIG. 6). The optical radiation is transmitted perpendicularly to the vessel axis. The ultrasonic transducer transmits ultrasonic waves in the direction opposite to that of the optical radiation. The ultrasonic signals are delivered to a processing unit.

Maschke in U.S. Pat. No. 7,289,842 describes an embodiment of a guidewire with both OCT and IVUS imaging transducers mounted upon it. The IVUS and OCT imaging mechanisms are located at different positions along the length of the guidewire. The sensor of the optical coherence tomography is directed to the side. The sensor of the Intravascular ultrasound imaging system is arranged in the front area of the catheter tip and directed to the side and/or diagonally forwards. Maschke describes also a display that jointly merges and displays the images processed by the OCT and by the IVUS processing devices. The center area of the display is a circular section of the image generated by the OCT, and the image generated by the IVUS in an outer area on the display. Maschke is aware that in order to generate an accurately detailed image of the artery it is worthwhile registering the images, producing the common image of the OCT and the IVUS imaging processing unit with each other. The technical term-“registration” designates images which feature the same phase relation. This ensures that the center image section and the outer image section surrounding it are displayed with the same phase so that the images coincide at the common edges.

US patent application 20080161696 to Schmitt describes a probe where OCT and IVUS can be performed simultaneously. The OCT and IVUS beams are parallel and opposite in direction. This arrangement of the beams facilitates proper co-registration of the images.

US patent application 20090043191 to Castella describes a catheter that incorporates an OCT system and an IVUS system for concurrent imaging of luminal systems. The system comprising a display that is configured to concurrently display signals received from each of the ultrasound transducer and the optical coherence tomography optical assembly in registration with each other.

US patent application 20080177183 to Courtney describes an imaging probe that combines IVUS and OCT to accurate co-registering of images during scanning a region of interest.

There is therefore a need for an imaging guidewire or probe that includes two or more imaging modalities so as to provide image data relating to two or more factors of a medical procedure.

### SUMMARY OF THE INVENTION

The present invention is an imaging guidewire or probe that includes two or more imaging modalities so as to provide image data relating to two or more factors of a medical procedure.

It is an objective of the present invention to enable ARIO to cross any type of occlusion in a safe manner.

It is also an objective of the present invention to enable the physician to see an image of the area to be treated and than an image of the area that has been treated.

It is another objective of the present invention to adopt the suitable imaging modality for each operation of the cutter. Forward looking IVUS for generating an image of the area to be treated and an optical imaging modality for imaging the treated area.

It is another objective of the present invention to incorporate in the imaging guidewire an optical imaging modality such as an OCT or a single fiber endoscope or a combination of both.

It is another objective of the present invention to discard the need for co-registration between IVUS and optical imaging.

It is another objective of the present invention to provide the physician with a cross sectional view of the blood vessel at the most distal longitudinal location that the cutter can reach in one stroke.

It is yet another objective of the present invention to provide the physician a view of inner wall of the bore so he can evaluate dissection, tissue prolapse surface smoothness, etc.

It is an additional objective of the present invention to allow the physician based on the images to decide how to proceed with the debulking process. For example, the physician can repeat the excision, or enlarge the bore or decide that the result is satisfactory and he can go on advancing the entire catheter.

It is an additional objective of the present invention to generate a full 3D image of the blood vessel. This image can be used for additional procedures, e.g., deployment of bio absorbable drug eluted stent.

According to the teachings of the present invention there is provided an imaging guidewire, comprising at its distal tip: (a) at least a first imaging sensor of a forward looking imaging system directed towards an area to be treated and configured to provide imaging data to an image processing system; (b) an optical imaging system directed towards an area that has already been treated and configured to provide imaging data of said treated area to said image processing system; and (c) at least one display device for outputting of images processed by said image processing system.

According to a further teaching of the present invention, said forward looking imaging system is an intravascular ultrasound imaging system and said first imaging sensor is configured for transmitting and receiving sound waves.

According to a further teaching of the present invention, said optical imaging system includes an optical fiber for directing and emitting light into said area that has already been treated and directing reflected light to said image processing system.

According to a further teaching of the present invention, said optical system is an optical coherence tomography imaging system.

According to a further teaching of the present invention, said optical system is a single fiber optic endoscope.

According to a further teaching of the present invention, said optical system is an optical coherence tomography fused with a single fiber optic endoscope.

There is also provided according to the teachings of the present invention, a method of operating an imaging guidewire during a medical procedure, the method comprising: (a) generating a first image of an area to be treated; (b) upon completion of at least a portion of the medical treatment, generating a second image of an area that has been treated; and (c) displaying at least said first and second images; wherein said first image can be generated by one or more imaging modalities and wherein said second image can be generated by one or more imaging modalities.

According to a further teaching of the present invention, said image is an endoscopic view of the blood vessel wall.

According to a further teaching of the present invention, said image is a cross sectional view of the blood vessel wall.

According to a further teaching of the present invention, said image is a fused image of endoscopic view and cross sectional view.

### BRIEF DESCRIPTION OF THE DRAWINGS

The invention is described herein, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more details than is necessary for a fundamental understanding of the invention, the description taken with the drawings making apparent to those skilled in the art how the several forms of the invention may be embodied in practice. Wherever possible, like reference numerals have been utilized to identify common elements throughout the figures.

In the drawings:

FIG. 1 is a view in longitudinal section of blood vessel where operation of the cutter perforates the blood vessel;

FIG. 2 is a view in longitudinal section of blood vessel with the cutter repositioned to enable safe excision of the plaque; and

FIG. 3 is a view in longitudinal section of blood vessel during the pulling back of the cutter.

### DESCRIPTION OF THE PREFERRED EMBODIMENTS

The present invention is an imaging guidewire or probe that includes two or more imaging modalities so as to provide image data relating to two or more factors of a medical procedure.

The principles and operation of an imaging guidewire or probe according to the present invention may be better understood with reference to the drawings and the accompanying description.

Briefly, the imaging guidewire of the present invention includes at its distal tip at least a first imaging sensor of a forward looking imaging system directed towards an area to be treated and configured to provide imaging data to an image processing system, an optical imaging system directed towards an area that has already been treated and configured to provide imaging data of said treated area to the image processing system and at least one display device for outputting of images processed by said image processing system. A method of operating the imaging guidewire of the present invention during a medical procedure includes the steps of:
1—Generating a first image of an area to be treated;
2—Upon completion of at least a portion of the medical treatment, generating a second image of an area that has been treated; and
3—Displaying at least the first and second images;

Wherein the first image can be generated by one or more imaging modalities and wherein the second image can be generated by one or more imaging modalities.

By way of introduction, the imaging guidewire of this application is an enhancement to the guidewire described in my U.S. Pat. No. 7,734,332 where the usage of one imaging modality incorporated in the guidewire is described. It is to be noted that this imaging guidewire can be incorporated also in various devices and other atherectomy or thrombectomy devices. This imaging guidewire can be used also for various diagnostic purposes. The imaging guidewire of this application includes two or more imaging modalities. The usage of two imaging modalities was described in prior art, specifically the combination of IVUS and OCT. However, the prior art is focused on the fusion of the two images received from both modalities into one image by co-registration.

Co-registration is not a simple task. The best co-registration is achieved when the beam of IVUS and the optical beam coincide, but it is not easy to accomplish this task because of construction limitations.

The present invention is configured, by non-limiting example for usage of both ultrasound imaging and optical imaging. A number of sensors, illustrated herein as two sensors by way of non-limiting example only, each configured to operate in one of the imaging modalities are deployed at the distal end of a torque tube. The sensors rotate together and can generate images at the same time, but the images obtained from both modalities are not merged into one image. Therefore, no co-registration is required, and the beams may be aimed in different directions. Preferably, the IVUS beam is directed forward towards the area to be treated, whereas the optical beam is directed towards the treated area. Each modality has a role during ARIO's operation. The IVUS that has a deep penetration depth is used prior to the operation of ARIO's cutter. More specifically before the cutter is pushed distally, for example into an occlusion. IVUS generates a cross sectional view of the blood vessel at the most distal location where the cutter can reach. It acts as an early warning system that will give the physician an alarm if there is a risk of perforation of the blood vessel. When such a warning is given the physician has the possibility to reposition the cutter in the blood vessel by using positioning balloons, so that the cutter will not encounter the vessel wall. The optical imaging is operated when the cutter is pulled proximally. At the time that the cutter is moving backwards, a bore in the plaque has already been created. The image is taken at the inner surface of the bore. The optical image used can be either OCT or a single fiber endoscope or a combination of both. OCT has high resolution and also has the ability to penetrate behind the bore surface, thus giving the physician additional information on the plaque composition. An endoscope has no penetration capability, but it can show the morphology of the inner surface. Recent developments of small diameter flexible endoscopes use a single fiber that can illuminate and also collect the image via the same fiber. Thus, the optical components that are used by the OCT can be used also by an endoscope.

Finally, it is possible to merge the image of OCT and the image of the endoscope into one image. Because the OCT and the endoscope are using the same optic elements, their radiation beams coincide to give an absolute co-registration. The optical image enables the physician to decide what further action he should take. He can either repeat the excision at same position of the cutter or he can enlarge the bore by reposition the cutter with the positioning balloons and than operate the cutter. During the pull back of the cutter a full 3D view derived out of the 2D cross sections can be generated similar to the manner it is done in existing pull-back OCT. The length of the 3D image created during the backward motion of the cutter equals the stroke of the cutter. ARIO's trajectory in the vessel is done piecewise. During each backward motion of the cutter a 3D image is recorded and eventually all the 3D images can be fused to a full 3D image of the blood vessel. A 3D view of the whole blood vessel can help the physician later with other procedures such as deploying No absorbable drug eluted stent, for healing the wound caused by excision.

It will be appreciated that for the purpose of non-limiting example only, the imaging guidewire of the present invention is illustrated in use an excision procedure. However, excision is only an example of any number of procedure for which the imaging arrangement of the present invention may be used to benefit. Therefore, it is to be understood that the term “excision” is used herein as a drawing specific term relating to the current drawing figures and such term is to be understood to include the broader meaning of the terms “treatment” and “medical procedure” and such terms should be understood to be interchangeable as used herein.

It is to be noted that although the physician is described here doing the operations, these operations can be executed automatically or semi-automatically via a console. For example repositioning the cutter in the blood vessel by inflating/deflating the balloons can be fully controlled by a computer using the input provided by the imaging modalities regarding the cross section view of the blood vessel.

An additional note is in regard to the sequence operation of the IVUS and the optical imaging: The IVUS operation is done before the forward movement of the cutter but it can continue to generate images during the entire forward movement. The optical imaging starts to generate images as soon as the cutter starts to move backwards. In ARIO no special means are needed because the reciprocation movements of forward and backward are inherent in ARIO's mechanism. It is clear that this concept can be used in other atherectomy devices where the forward and pull back motions can be added to their procedure.

It will be appreciated that the reciprocating movement of the imaging guidewire is not mandatory. There are other procedures such as, but not limited to, laser atherectomy where the excision can be done distally to the imaging guidewire tip. In this case an image of the area to be treated is taken before the excision, than the imaging guidewire is advanced forward rather than backwards and finally an image of the treated area is taken after the excision.

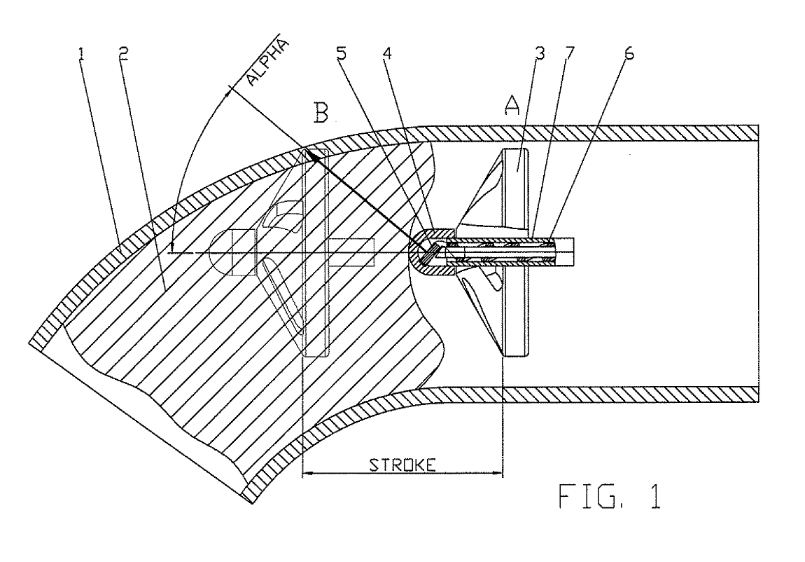

Referring now to the drawings, FIG. 1, a main goal of incorporating the imaging capability in the device is to minimize the risk of damage to healthy surrounding portions of the vessel wall. FIG. 1 shows a longitudinal cross section of blood vessel 1, that has an occlusion 2. It is to be noted that the occlusion 2 shown in the drawing is a total occlusion. Total occlusions pose a bigger challenge for crossing it because the physician has no information as to how the blood vessel progresses distally, e.g., if it is curved; therefore, there is a higher risk of perforation. It is obvious that the present invention is applicable also for partial occlusions. ARIO's procedure requires that first the physician inserts the imaging guidewire up to the site of the occlusion. Then the catheter with the working head 3 at its distal tip is advanced over the guidewire until the catheter is stopped by cap of the imaging guidewire 4. This position is designated “A” in the drawing. The operation of ARIO's mechanism is characterized by a combined movement of the cutting head, it performs a longitudinal reciprocating (forward and backward) movement combined with a unidirectional rotation. The maximum forward movement of the cutter is designated “STROKE” as shown in the drawing. For the case depicted in the drawing the forward movement of the cutter to position “B” will cause penetration of the upper part of the cutter into the vessel walls resulting in blood vessel perforation, a situation that is absolutely forbidden.

A first imaging modality, IVUS, can penetrate the plaque and generate a cross sectional view at position “B”. The sound waves pass through cap 4 that includes a window that is transparent to sound waves. The sound beam is represented by the arrow in the drawing. Following penetration of the plaque by the sound waves, a unique representation of the vessel as well as the plaque composition is obtained. The penetration depth required and the forward looking angle designated “ALPHA” in the drawing is dependent on the geometry of the cutter and the location of ultrasonic sensor 5. As a non limiting example, for a cutter that has an OD of 2.7 mm and a stroke of 2.6 mm the required penetration depth is 2.1 mm and the forward looking angle is 40 degrees. IVUS is a suitable imaging modality because it can penetrate more than 2.1 mm. The resolution of the image is around 100 um. The image generated by IVUS sometimes needs an expert for segmentation. However, manual segmentation is time-consuming and susceptible to observer variance. It is preferred to use an automatic method to segment the plaque and the vessel. This information serves as input to the computer for positioning the balloons. Also shown in the drawing are electrical conduits 6 for delivering power to the ultrasonic transducer and sending electrical signals to the processor device (not shown here). In order to have a 360 degrees image, the IVUS sensor must be rotated. Usually the rotation is at 1800 RPM, in order for a video image to be generated. IVUS is rotated by a flexible torque cable 7 that must provide a stable revolution rate of the assembly. If the torque cable does not have enough torsion stiffness, the quality of the image is degraded. This phenomenon is known as NURD- Non-uniform Rotation Distortion.

It is clear that the items described above are only a part of the IVUS system that is located at the proximal end of the imaging guidewire, outside the patient body. At the proximal end there are units such as a slip-ring that couples the rotating electrical conduits to a processor device that generates the image, a display, etc. These units are well-known in art and are not a part of the present invention.

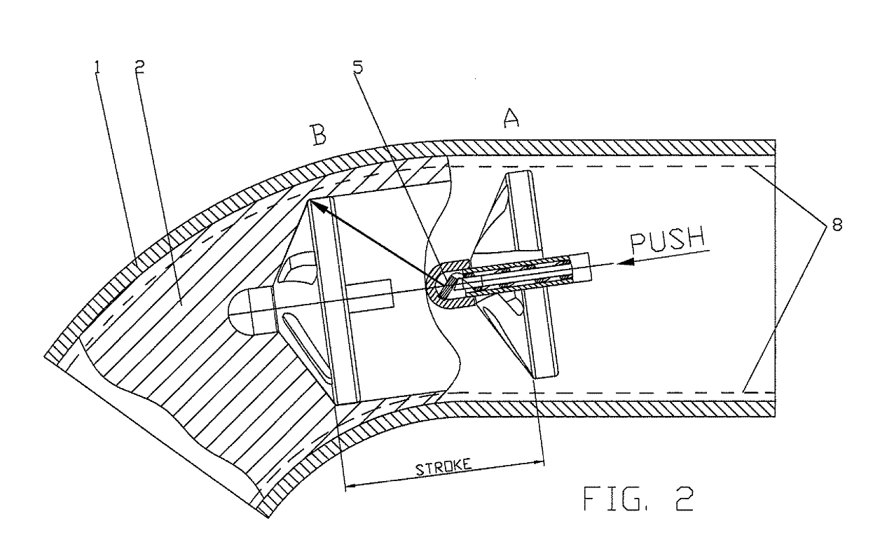

FIG. 2 illustrates the situation after the physician repositions the cutter in the blood vessel. ARM includes positioning balloons (not shown) each of which is inflated/deflated separately and therefore the cutter can be positioned in the vessel at substantially any spatial position. It is shown in this figure that the cutter is pushed a stroke forward without perforating the vessel walls. For restoring sufficient blood flow in the lumen it is not required to open the vessel to its full inside diameter. The physician can define to the system an imaginary border line 8 and only the plaque material inside this zone will be removed. The border line 8 diameter is smaller then the inside diameter of the blood vessel 1, thus reducing the risk of blood vessel perforation while operating ARIO. It is to be noted here also, that although the physician is described here doing the operations, these operations can be executed automatically or semi-automatically under the supervision of the physician. For example, repositioning the cutter in the blood vessel by inflating/deflating the balloons can be fully controlled by a computer following inputs of the cross section view of the blood vessel provided by the IVUS imaging modality.

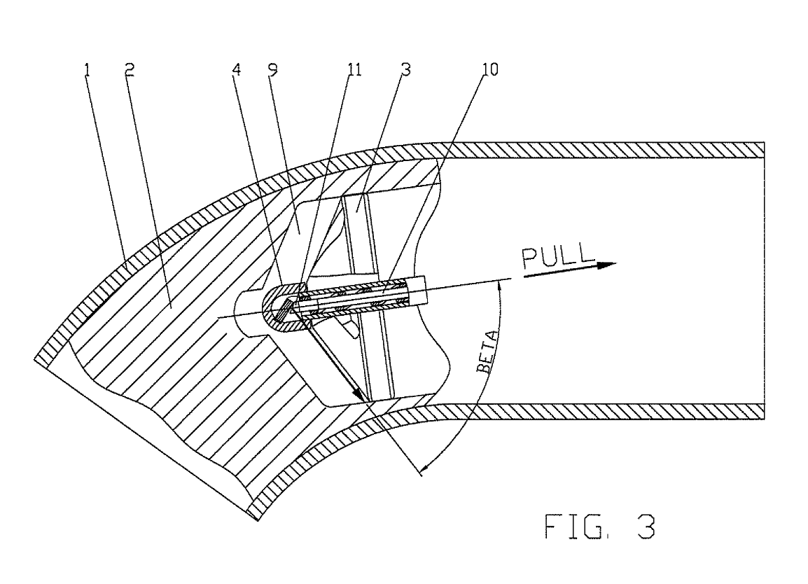

FIG. 3 illustrates the situation of ARIO as the cutter is pulled back. A bore 9 has already been formed in the plaque by the forward stroke illustrated in FIG. 2. A second imaging modality engages now into operation. This imaging modality provides the physician with an image that shows the outcome of the excision of the occlusion. It provides him with a view of inner wall of the bore so he can evaluate dissection, tissue prolapse, surface smoothness, etc.

While this imaging modality is, not required to deeply penetrate into the plaque, it is preferable that it have good resolution. Optical modality has these characteristics. The components of the optical modality are: single mode fiber 10 and an angled tip lens 11. The angled tip lens 11 and the fiber direct and emit light into the excised area and direct reflected light from the excised area to an image processing device. Light passes through cap 4 that includes a window that is transparent to optical radiation. The optic radiation that is represented as an arrow in the drawing is pointing backwards and its angle to the cutter axis is designated in the drawing as “BETA”. This angle is dependent on the geometry of the cutter. In ARIO the angle is 90 degrees for a flat cutter and less than 90 degrees for a cone shaped cutter. As a non limiting example, in the drawing “BETA” is 60 degrees. It is to be noted that it is possible that in other devices such as atherectomy or thrombectomy devices or other procedures the optical beam can be aimed forward.

There are two optic imaging modalities that can be used. The first option is OCT that has a resolution of 5-20 um and a penetration depth of 1 mm. The second option is a single fiber endoscope that has no penetration capability but has good resolution. Each of these optic modalities can be used. There is also the possibility to fuse the images of both modalities into one image. Because the OCT and the endoscope are using the same optic elements, their radiation beams coincide to give an absolute co-registration.

It will be appreciated that the features described above are only a part of the optical modality system that is located at the proximal end of the imaging guidewire, outside the patient body. At the proximal end there are units such as a FORT (Fiber Optic Rotary Joint) that couples the rotating fiber to a processor device, a processor device that generates the image, a display etc. These units are well known in art and are not a part of the present invention.

It is to be noted that the IVUS and the optic modality can generate images all the time the guidewire is rotated. However, meaningful images are retrieved only during the times described above. The images generated can be presented on separate displays, on two or three regions of the same display or on one display by swapping the images.

It will be appreciated that the above descriptions are intended only to serve as examples and that many other embodiments are possible within the spirit and the scope of the present invention. It should be noted that is expected that during the life of this patent many relevant minimally invasive imaging techniques that can generate an image of the blood vessel will be developed. It is also possible that images can be generated by fusion of two or more modalities. The scope of the terms “image” and “imaging” is intended to include all such new technologies a priori.

What is claimed is:

1. An imaging guidewire, comprising at its distal tip:
(a) at least a first imaging sensor of a forward looking imaging system directed towards an area to be treated and configured to provide imaging data to an image processing system;
(b) an optical imaging system directed towards an area that has already been treated and configured to provide imaging data of said treated area to said image processing system; and
(c) at least one display device for outputting of images processed by said image processing system.

2. The imaging guidewire of claim 1, wherein said forward looking imaging system is an intravascular ultrasound imaging system and said first imaging sensor is configured for transmitting and receiving sound waves.

3. The imaging guidewire of claim I, wherein said optical imaging system includes an optical fiber for directing and emitting light into said area that has already been treated and directing reflected light to said image processing system.

4. The imaging guidewire of claim 3, wherein said optical system is an optical coherence tomography imaging system.

5. The imaging guidewire of claim 3, wherein said optical system is a single fiber optic endoscope.

6. The imaging guidewire of claim 3, wherein said optical system is an optical coherence tomography fused with a single fiber optic endoscope.

7. A method of operating an imaging guidewire during a medical procedure, the method comprising:
(a) generating a first image of an area to be treated;
(b) upon completion of at least a portion of the medical treatment, generating a second image of an area that has been treated; and
(c) displaying at least said first and second images;
wherein said first image can be generated by one or more imaging modalities and wherein said second image can be generated by one or more imaging modalities.

8. The method of claim 7, wherein said image is an endoscopic view of the blood vessel wall.

9. The method of claim 7, wherein said image is a cross sectional view of the blood vessel wall.

10. The method of claim 7, wherein said image is a fused image of endoscopic view and cross sectional view.
